(19) United States
(12) Patent Application Publication
(10) Pub. No.: US 2023/0148953 A1
(43) Pub. Date: May 18, 2023

(54) MANDIBULAR ADVANCEMENT DEVICE FOR PREVENTING SLEEP APNEA

(71) Applicant: Hyeran CHOO, Palo Alto, CA (US)

(72) Inventor: Hyeran CHOO, Palo Alto, CA (US)

(21) Appl. No.: 17/986,798

(22) Filed: Nov. 14, 2022

(30) Foreign Application Priority Data
Nov. 15, 2021 (KR) 10-2021-0156568

(51) Int. Cl. A61B 5/00; A61F 5/56

(52) U.S. Cl. CPC A61B 5/4818; A61F 5/566

(57) ABSTRACT

The present disclosure discloses a mandibular advancement device for preventing sleep apnea that moves a mandible forward to prevent sleep apnea. The mandibular advancement device includes: a first hook to which a first end of an elastic member providing a mandible traction force for advancing the mandible can be connected and that supports the elastic member; an upper frame that can be mounted on upper teeth to distribute a traction support force, which is applied to the first hook by the elastic member, to a maxilla; a second hook to which a second end of the elastic member can be connected and that transmits the mandible traction force applied by the elastic member to the mandible at a distal location in comparison to the first hook; and a lower frame that can be mounted on lower teeth under the upper frame to distribute the mandible traction force, which is applied to the second hook, to the mandible.

### CROSS-REFERENCE TO RELATED APPLICATIONS

This application claims the priority of Korean Patent Application No. 10-2021-0156568 filed on Nov. 15, 2021, in the Korean Intellectual Property Office, the disclosure of which is incorporated herein by reference.

### BACKGROUND OF THE INVENTION

### Field of the Invention

The present disclosure relates to a mandibular advancement device for preventing sleep apnea and, in detail, a device for advancing the mandible to prevent sleep apnea symptoms such as obstructive sleep apnea.

### Description of the Related Art

Sleep apnea, which is a sleep breathing disorder accompanied with frequent waking due to stop or reduction of breathing by repetitive obstruction of the upper respiratory tract during sleep, is a disease that may cause not only excessive daytime sleepiness or a recognition disorder, but hypertension, cardiovascular diseases, abnormal glycometabolism, etc. if it is not treated.

Sleep apnea can be classified into obstructive sleep apnea that occurs due to obstruction the respiratory tract in the oral cavity even though there is an attempt to breathe and central sleep apnea that shows temporal stop of an attempt itself to breathe. Hypoventilation means a situation in which breathing is not completely stopped, but the amount of breath reduces under a half, whereby concentration of oxygen in blood decreases.

When two or more of five symptoms of excessive daytime sleepiness, choking during sleep, repetitive waking during sleep, not feeling well even after sleep, and daytime fatigue and concentration reduction are shown and a sleep breathing disorder is shown in a polysomnograph test, obstructive sleep apnea is diagnosed. The sleep breathing disorder means the case in which apnea or hypoventilation occurs 5 or more times per hour.

It has been known that over 90% of such sleep apnea is obstructed sleep apnea. A reason of obstructive sleep apnea is narrowing of the upper respiratory tract during sleep. When the pharyngeal airway is narrowed by hypertrophy of the velum and the uvula and dilatation of the tonsils and the tongue, etc., which can be frequently observed from patients with sleep apnea, air is difficult to move over the pharyngeal airway in breathing, so more effects are required to breathe in.

It does not matter when the respiratory tract dilates due to these efforts, but if the respiratory tract does not dilate due to fatigue of the pharyngeal dilator muscles, etc., sleep apnea occurs. That is, sleep apnea occurs when the pharyngeal airway narrows and the respiratory tract does not dilate.

Sleep apnea described above may cause metal/physical complications.

In detail, sleep apnea may cause various psycho neurological complications by causing awakening during sleep and segmenting sleep, and it has been known that, for example, other than fatigue, a personality change, a recognition ability disorder, deterioration of driving ability, etc. may occur with high frequency.

Further, it was reported that arrhythmia occurs 2 to 4 times in patients with serious sleep apnea in comparison to normal people and the relationship between sleep apnea and hypertension has been well known. Furthermore, it has been known that sleep apnea is highly connected with arteriosclerosis and cardiac insufficiency too and increases frequency of crebrovascular diseases.

As described above, sleep apnea is accompanied with severe snoring during sleep, a sleep disorder such as lethargy, etc. and frequently causes apnea during sleep, so it is a disease that may various cardiovascular and pulmonary vascular diseases due to hypoxemia.

FIG. 1 is a view showing a “DENTAL APPLIANCE FOR TREATMENT OF SNORING AND OBSTRUCTIVE SLEEP APNEA” disclosed in U.S. Pat. No. 5,868,138.

FIG. 1 is an intraoral device for maintaining a respiratory tract at the upper pharyngeal airway of a patient. The intraoral device includes an upper member 11, a lower member 12, a connector 13, and slots 14. When the connector 13 is selectively inserted in any one of the slots 14, the lower member 12 may be maintained at a position protruding forward further than the upper member 11.

In the device shown in FIG. 1, since the lower member 12 protrudes forward further than the upper member 11, a respiratory tract is secured of a user and snoring is prevented in sleep. Further, the upper member 11 and the lower member 12 are manufactured to fit the sets of teeth of a patient, but the device is large, complicated, and inconvenient to use.

As another example of treatment device for snoring and sleep apnea, a device including an upper splint in which upper teeth are inserted, a lower splint in which lower teeth are inserted, and a rod connecting the upper splint and the lower splint, in which an end and another end of the rod are hinged to the upper splint and the lower splint, respectively, and the lower splint is moved forward by operating the rod is also used.

### SUMMARY OF THE INVENTION

An objective of the present disclosure is to provide a mandibular advancement device for preventing sleep apnea, in detail, obstructive sleep apnea (OSA), the device bang able to appropriately adjust and advancement amount of the mandible.

An aspect of the present disclosure relates to a mandibular advancement device for preventing sleep apnea that moves a mandible forward to prevent sleep apnea. The mandibular advancement device includes: a first hook to which a first end of an elastic member providing a mandible traction force for advancing the mandible can be connected and that supports the elastic member; an upper frame that can be mounted on upper teeth to distribute a traction support force, which is applied to the first hook by the elastic member, to a maxilla; a second hook to which a second end of the elastic member can be connected and that transmits the mandible traction force applied by the elastic member to the mandible at a distal location in comparison to the first hook; and a lower frame that can be mounted on lower teeth under the upper frame to distribute the mandible traction force, which is applied to the second hook, to the mandible.

The mandibular advancement device has the following effects.

First, according to the present disclosure, since it is possible to easily and appropriately adjust an advancement amount of a mandible, it is effective in preventing sleep apnea, in detail, obstructive sleep apnea (OSA) of not only adults, but children.

Second, according to a fixed-type mandibular advancement device, for example, an archwire-type mandibular advancement device that is fixed to upper teeth and lower teeth, an occlusal clearance is not required, so patients can more comfortably use the device.

Third, when a mandibular advancement device, that is, a hybrid-type mandibular advancement device having an archwire type frame (fixable frame) that is mounted on one of upper teeth and lower teeth and a removable splint-based frame (removable frame) is mounted on another one is applied to the inside of an oral cavity, the occlusal clearance can be greatly reduced in comparison to existing devices and the splint type frame can be used in a removable type, so it is possible to more comfortably use the device.

Fourth, the fixable frame that is applied to the present disclosure can be applied to young child patents with primary dentition or transitional dentition from whom it is very difficult to an optimal maintaining force using an acrylic removable frame due to anatomical limitation of primary teeth (lack of undercut that is important to maintain an acrylic device). Further, the fixable frame is effective to young child patents because its cooperation level in use of device is low in comparison to a detachable (removable frame).

Fifth, the hybrid-type mandibular advancement device is helpful to not only patients with obstructive sleep apnea (OSA) requiring vertical occlusion, but patients with temporomandibular disorder (TMD).

Sixth, according to the present disclosure, a user has only to carry an elastic member such as an elastic rubber band or an elastic rod, the device can be very conveniently carried in comparison to acrylic removable devices having large volume in the related art. Further, it is possible to easily titrate an advance amount of a mandible by applying different elastic members.

Seventh, the present disclosure has an excellent effect to young child patient with transitional dentition, can maintain a clearance in eruption of permanent teeth. In particular, the fixable frame does not require a remake or correction process in eruption of permanent teeth and can sufficiently maintain teeth while dentition changes. Further, the fixable frame prevents deterioration of maintaining force in comparison to removable frames and it can be omitted to make a space for a permanent tooth that is slowly erupted.

Eighth, existing mandibular advancement devices are for only adults and there is not mandibular advancement device for children, but the present disclosure can achieve various functional priorities for not only adult patients with OSA who prefer the convenience and comfortableness of a fixable frame, but child patients with OSA.

### BRIEF DESCRIPTION OF THE DRAWINGS

The features and advantages of the present disclosure will be more clearly understood from the following detailed description of embodiments of the present disclosure with the accompanying drawings to be described below, in which:

FIG. 1 is a view showing a device for preventing sleep apnea of the related art;

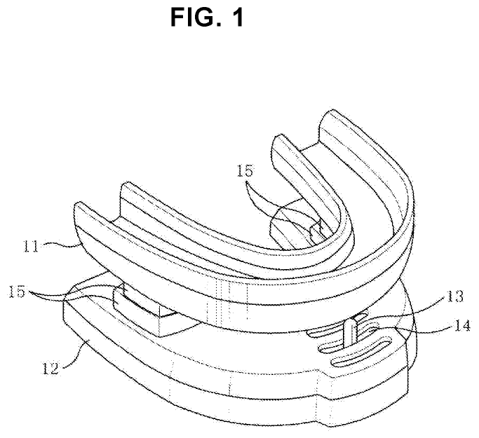

FIGS. 2A and 2B are views showing a first embodiment of a mandibular advancement device for preventing sleep apnea according to the present disclosure;

FIGS. 3A and 3B are views showing a second embodiment of a mandibular advancement device for preventing sleep apnea according to the present disclosure;

FIGS. 4A and 4B are exemplary views showing the state in which an elastic member is connected to a splint-type removable mandibular advancement device shown in FIGS. 3A and 3B;

FIGS. 5A and 5B are views showing the state in which the mandibular advancement device for preventing sleep apnea shown in FIGS. 2A and 2B has been installed on teeth;

FIGS. 6A and 6B are views showing the state in which a third embodiment of a mandibular advancement device for preventing sleep apnea according to the present disclosure has been installed on teeth;

FIGS. 7A and 7B are views showing the state in which a fourth embodiment of a mandibular advancement device for preventing sleep apnea according to the present disclosure has been installed on teeth;

FIGS. 8A and 8B are views showing the state in which a fifth embodiment of a mandibular advancement device for preventing sleep apnea according to the present disclosure has been installed on teeth;

FIGS. 9A and 9B are views showing the state in which a sixth embodiment of a mandibular advancement device for preventing sleep apnea according to the present disclosure has been installed on teeth;

FIGS. 10A and 10B are views showing the state in which a seventh embodiment of a mandibular advancement device for preventing sleep apnea according to the present disclosure has been installed on teeth;

FIGS. 11A and 11B are views showing the state in which an eighth embodiment of a mandibular advancement device for preventing sleep apnea according to the present disclosure has been installed on teeth;

FIGS. 12A and 12B are views showing the state in which a ninth embodiment of a mandibular advancement device for preventing sleep apnea according to the present disclosure has been installed on teeth;

FIGS. 13A and 13B are views showing the state in which a tenth embodiment of a mandibular advancement device for preventing sleep apnea according to the present disclosure has been installed on teeth;

FIGS. 14A and 14B are views showing the state in which the mandibular advancement device for preventing sleep apnea shown in FIGS. 3A and 3B has been installed on teeth;

FIG. 15 is a view showing the state in which an eleventh embodiment of a mandibular advancement device for preventing sleep apnea according to the present disclosure has been installed on teeth; and

FIGS. 16A and 16B are another embodiments of an upper archwire and a lower archwire of the mandibular advancement device for preventing sleep apnea according to the present disclosure.

### DETAILED DESCRIPTION OF THE INVENTION

Hereafter, an exemplary embodiment that can achieve the objectives of the present disclosure in detail is described with reference to the accompanying drawings. In the description of the embodiment, the same components are given the same names and reference numerals and are not repeatedly described.

Terms used herein are used to describe embodiments of the present disclosure and it is to be understood that when one element is referred to as being “connected to” or “coupled to” another element, it may be connected directly to or coupled directly to another element or be connected to or coupled to another element with another element therebetween, including indirect connection.

It will be further understood that the terms “comprises” or “have” used in this specification, specify the presence of stated features, steps, operations, components, parts, or a combination thereof, but do not preclude the presence or addition of one or more other features, numerals, steps, operations, components, parts, or a combination thereof.

An appropriate amount of advancement (titration) is a very important factor in a mandibular advancement device (MAD) for sleep apnea, for example obstructive sleep apnea. The mandibular advancement amount may be adjusted through a titration mechanism, depending on how a patient reacts to a predetermined mandibular advancement amount. When the advance amount is large, a patient feels uncomfortable, and when the advance amount is too small, there will be not improvement of OSA, so appropriate adjustment is required and this advancement amount adjustment process is called ‘titration’.

When the MAD for an OSA patient is an entirely removable device, for example, when a removable acrylic device is applied to both of upper and lower teeth, this removable device is loosened over time, so periodic remake or relining is required, it is difficult to apply larger maintaining force, it is difficult to carry the device in traveling, and inconvenience may be increased by an occlusal clearance. Further, at present, there is no MAD for children approved by FDA.

The present disclosure relates to a mandibular advancement device for sleep apnea that is convenient to use, can be applied to children with a low treatment cooperation level, and is more comfortable in use.

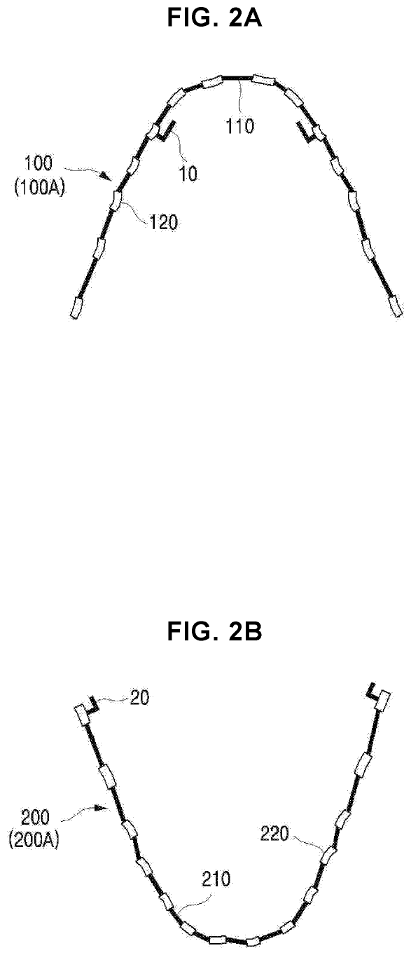

First, referring to FIGS. 2A to 5B, the present disclosure, which is a device for preventing sleep apnea symptoms, that is, a device for improving, attenuating, or treating sleep apnea, is a mandibular advancement device for sleep apnea that advances a mandible to prevent sleep apnea.

The mandibular advancement device for sleep apnea (hereafter, referred to as an ‘MAD’) according to the present disclosure includes a first hook 10 supporting an elastic member 30, an upper frame 100 that can be mounted on upper teeth UT, a second hook 20 connected to the elastic member 30, and a lower frame 200 being able to be mounted on lower teeth LT. That is, the MAD may be implemented in an intraoral device type.

The elastic member 30 is an element for providing a force for advancing a mandible 2, that is, a mandible traction force (mandible advancement force) to the mandible 2. A first end of the elastic member 30 may be connected to the first hook 10 and a traction support force (force supporting the mandible traction force) applied to the first hook 10 is transmitted to a maxilla 1.

The upper frame 100 is mounted on the upper teeth UT to distribute the force (traction support force) applied to the first hook 10 by the elastic member 30 to the maxilla 1. In more detail, the traction support force applied to the first hook 10 is distributed to the upper teeth by the upper frame 100 and distributed to the maxilla 1. Accordingly, concentration of traction support force can be reduced or prevented at the portion where the first hook 10 is positioned.

A second end of the elastic member 30 is connected to the second hook 20. The second hook 20 transmits the mandible traction force by the elastic member 30 to the mandible 2 at a distal location further than the first hook 10.

Next, the lower frame 200 is mounted on the lower teeth LT under the upper frame 10 to distribute the mandible traction force applied to the second hook 20 to the mandible 2. In more detail, the mandible traction force applied to the second hook 20 is distributed to the lower teeth LT by the lower frame 200 and distributed to the mandible 2. Accordingly, concentration of traction force can be reduced or prevented at the portion where the second hook 20 is positioned.

In embodiments of the present disclosure, the upper frame 100 fastens the upper teeth UT and the lower frame 200 fastens the lower teeth LT. To this end, the upper frame 100 may be mounted on the upper teeth UT in a fixed type or a removable type and the lower frame 200 may be mounted on the lower teeth in a fixed type or a removable type.

For reference, the upper teeth UT are a plurality of teeth at the maxilla 1 and the lower teeth LT are a plurality of teeth at the mandible 2.

The first hook 10 may be disposed at the upper frame 100 and the second hook 20 may be disposed at the lower frame 200. Of course, the first hook 10 may be directly fixed to a tooth itself of the upper teeth 1, that is, front tooth parts such as both canines UC. The second hook 200 may be directly fixed to a tooth itself of the lower teeth LT, that is, the posterior teeth of the lower teeth such as molars at both sides. The elastic member 30 may be removably connected to the first hook 10 and the second hook 20.

The elastic member 30, for example, may be a rubber band 30a, a spring-shaped elastic rod 30b, or an elastic strap. For example, the elastic rod 30b may include a spring body 31 and hook holes 32a and 31b at both ends of the spring rod. Accordingly, the mandible advancement force and advancement amount can be adjusted in accordance with the length of the elastic member, and an elastic member having an appropriate length can be selected, depending on the condition of patients.

The upper frame 100 may include an upper archwire 100A mounted on the upper teeth 1 in a fixed type and/or an upper splint 100b in which the dental arch of the upper teeth 1 is fitted.

For example, the upper frame 100, as in the example shown in FIG. 2A, may include an upper archwire 100A mounted (attached) in a fixed type to the maxillary arch (superior dental arch). The first hook 10 may be may be disposed in both front teeth area of the upper archwire 100A, for example, a canine area adjacent to canines UC.

As a more detailed example, the upper archwire 100A may include a lingual archwire fixed to the lingual side of the upper teeth UT and the lingual archwire 110 may be fixed to the lingual side of the maxillary arch by wire fixing member 120 such as wire fixing pads or wire fixing bases that are fixed to tooth surfaces.

Of course, the upper archwire may be manufactured in a type that is installed on the labial side of upper teeth, but the lingual archwire is better in terms of aesthetic/mental aversion. The upper archwire 100A may be manufactured integrally with the wire fixing members 120 in a patient-customized type through 3D printing, etc., and then fixed to the upper teeth UT of the patient through resin. The second hook 10 may be formed integrally with the upper archwire 100A through 3D printing or metal bonding such as soldering.

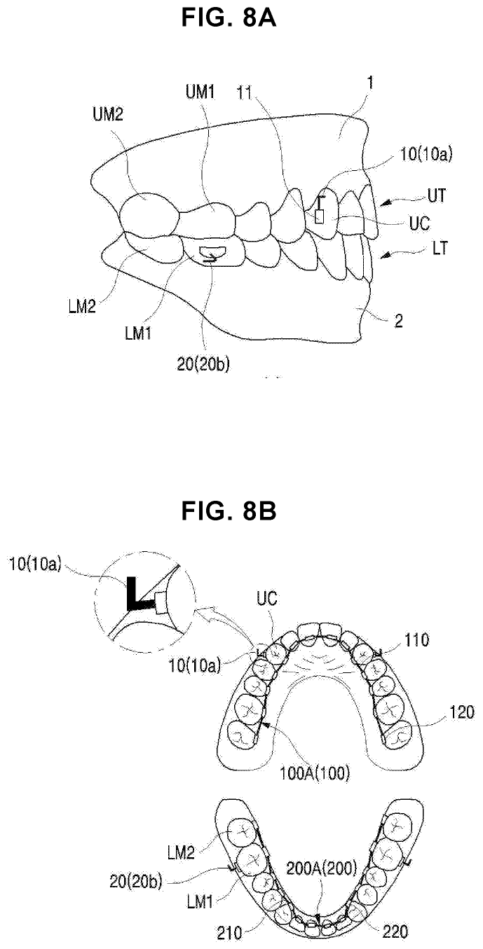

The first hook 10 may be formed on the lingual side or the palatal side of the upper archwire 110. The palatal side includes the gums direction. The first hook 10 may be formed on a buccal side on the upper archwire 100A across the embrasure between a canine and a first premolar UP1 (see FIGS. 8A and 8B).

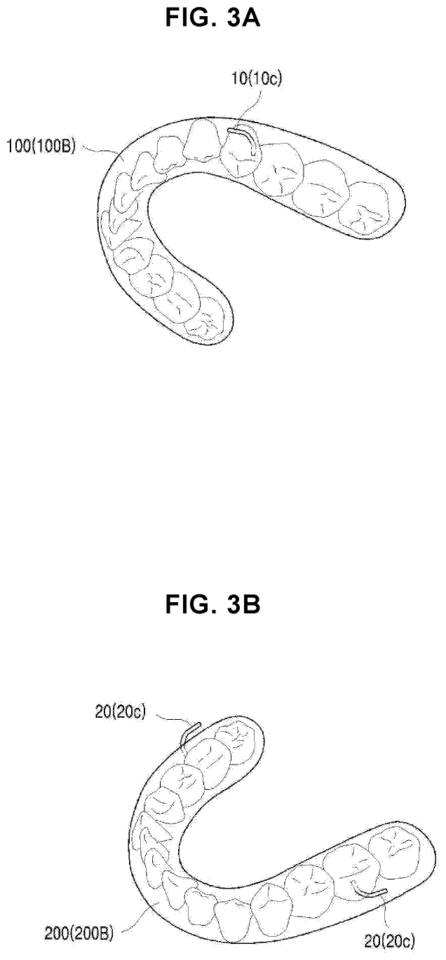

The upper frame 100, as in the example of FIG. 3A, may include an upper splint 100B in which the upper teeth 1 are fitted. A mouthpiece-type removable frame may be applied as the upper frame of the mandibular advancement device according to the present disclosure. In this case, the first hook 10 may be disposed in both front teeth areas (both side areas of a front teeth seat section) of the upper splint 100B, but is not limited thereto and may be disposed at other positions.

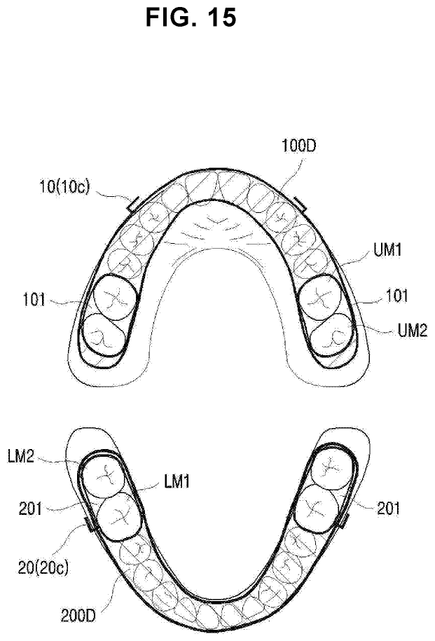

The upper splint 100B, as in the example 100D shown in FIG. 15, may be modified in a shape in which the occlusion sides (bottom sides) of molar seat sections in which the posterior teeth of the upper teeth, particularly, molars UM1 and UM2 are fitted are bored such that occlusal surfaces of the upper molars are exposed.

The lower frame 200 may include at least one of a lower archwire 200A on which the arch of the lower teeth 1 is mounted in a fixed type and a lower splint 200B in which the mandibular arch is fitted.

For example, the lower frame 200, as shown in FIG. 2B, may include a lower archwire 200A that is mounted (attached) on the mandibular arch. It is advantageous in terms of adjustment of a mandibular advancement amount that the second hook 20 is disposed posterior teeth area at both sides of the lower archwire 200A, for example, as distal teeth area as possible of the lower teeth such as molars, in more detail, the area of a first molar LM1 or a second molar LM2.

As a more detailed example, the lower archwire 200A may include a lingual archwire the lingual side of the lower teeth 1 and the lingual archwire 210 may be fixed to the lingual surface of the lower teeth by wire fixing members 220.

Of course, the lower archwire 200A may be applied in a type that is attached to the labial side of the lower teeth, but the lingual archwire is better in terms of aesthetic/mental aversion. The lower archwire 100A may also be manufactured integrally with the wire fixing members 220 through various methods such as 3D printing and then fixed to the upper teeth of a patient through resin. The second hook 20 may be formed integrally with the lower archwire through 3D printing or metal bonding such as soldering.

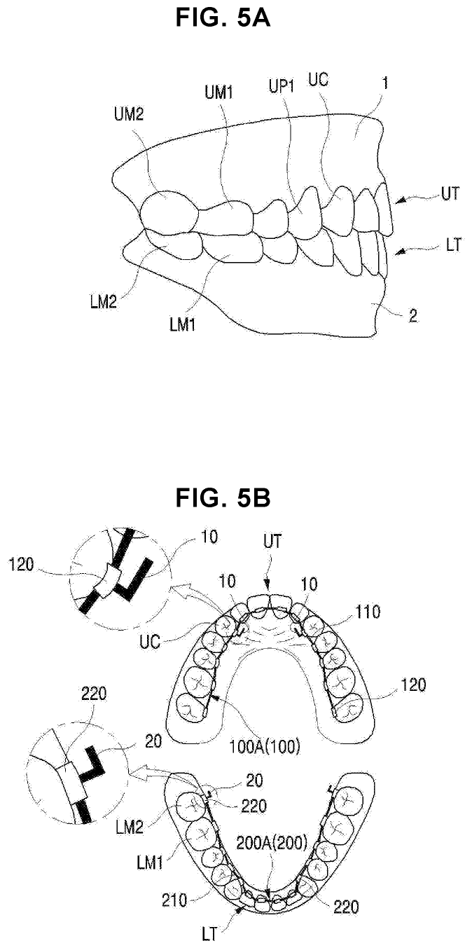

The second hook 200 may include a lingual hook formed on the lingual side of the lower archwire 200A or a buccal hook (see FIGS. 5A and 5B) across the embrasure of posterior teeth of the lower archwire 200A, for example, the area between a first molar LM1 and a second molar LM2.

The lower frame 200, as in the example of FIG. 3B, may include a lower splint 200B in which the lower teeth LT are fitted. That is, a mouthpiece-type removable frame may be applied as the lower frame of the mandibular advancement device according to the present disclosure. In this case, the second hook 20 is better to be disposed in the posterior teeth areas at both sides, in detail, molar areas of the lower splint 200B. That is, the second hook 20 is better to be disposed in the posterior teeth seat sections at both sides, preferably, molar seat sections of the lower splint 200B.

The lower splint 200B, as in the example 200D shown in FIG. 15, may be modified with the occlusion sides (top sides) of molar seat sections in which the lower molars are fitted are bored such that occlusal surfaces of the lower molars LM1 and LM2 are exposed.

FIGS. 2A and 2B are an embodiment (first embodiment) having fixable frames, that is, archwire-type upper frame and lower frame that are attached to teeth and FIGS. 3A and 3B are embodiments (second embodiment) having removable frames, that is, splint-type upper frame and lower frame in which teeth are fitted, but any one of the upper frame and the lower frame may be an archwire-type fixable frame and the other one may be a splint-type removable frame.

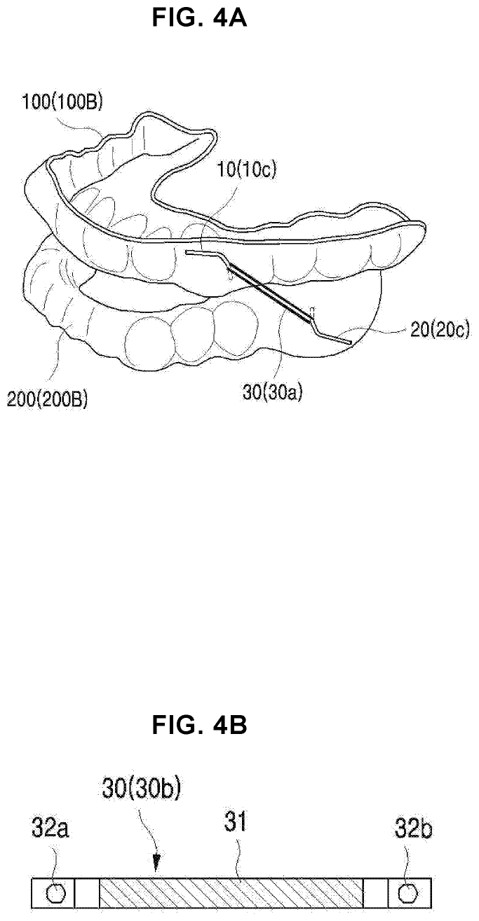

FIGS. 4A and 4B show examples in which the upper splint 100B and the lower splint 200B are connected by the elastic member 300, for example, the rubber band 30a, and mandibular advancement is implemented by connecting a first hook and a second hook through the elastic member 30 also in other embodiments of the present disclosure.

FIGS. 5A and 5B are views showing the state in which the fixable (attachable) upper frame (upper archwire 100A) and the fixable lower frame (lower archwire 200A) shown in FIGS. 2A and 2B are mounted on the upper teeth UT and the lower teeth LT. In other words, the upper archwire 100A is fixed to the lingual side of the upper teeth UT, the lower archwire 200A is fixed to the lingual side of the lower teeth LT, the first hook 10 is integrally formed on the lingual side of the upper archwire 100A, and the second hook 20 is integrally formed on the lingual side of the lower archwire 200A.

In more detail, it is advantageous in terms of titration of an advancement amount that the first hook 10 is positioned in the area of a canine (UC) and the second hook 20 is positioned in the most distal tooth area, for example, the area of a second molar (LM2), but they are not limited thereto. For example, the first hook 10 may be positioned at the lingual side of the area of a lateral incisor or a first premolar UP1 and the second hook 20 may be positioned at the lingual side of the area of a first molar LM1.

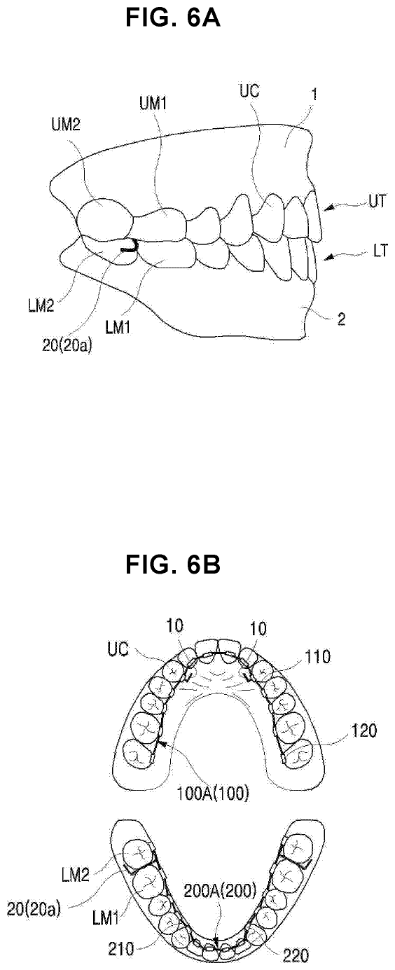

FIGS. 6A and 6B show another embodiment (third embodiment) of the MAD and the embodiment of FIGS. 6A and 6B includes the upper frame 100 (100A), the lower frame 200 (200A), and the first hook 10 that have the same structures as those of the example shown in FIGS. 5A and 5B, but a second hook 20a connected to the lower frame, that is, the lower archwire is positioned on the buccal side of the lower teeth.

In more detail, the second hook 20a is formed on the buccal side of the lower archwire 100A, that is, the lingual archwire 110 across the embrasure of posterior teeth, for example, the area between a first molar LM1 and a second molar LM2. Accordingly, the elastic member 30 described above can be connected from the buccal side of the lower teeth to the lingual side of the upper teeth.

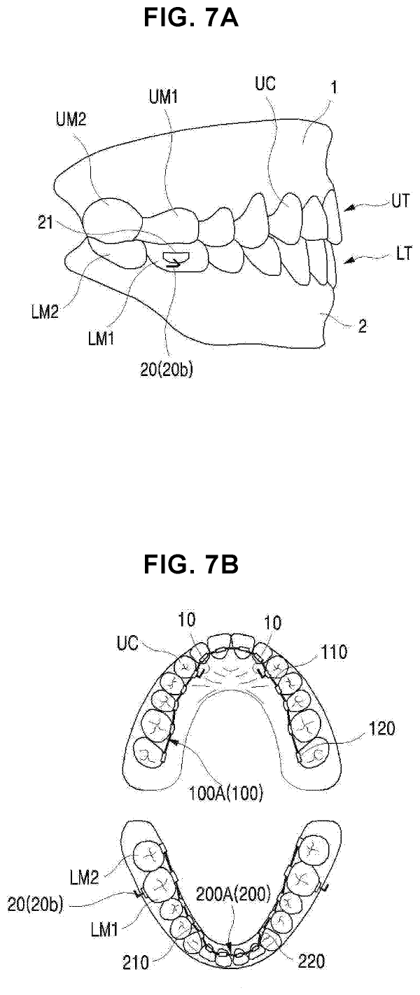

FIGS. 7A and 7B show a fourth embodiment of the MAD and the embodiment of FIGS. 7A and 7B includes the upper frame 100 (100A), the lower frame 200 (200A), and the first hook 10 that have the same structures as those of the example shown in FIGS. 5A and 5B, but the fourth embodiment has a second hook 20b that is fixed (attached) to a specific tooth of the lower teeth.

In more detail, the second hook 20b is fixed to the buccal side of the posterior teeth section, for example, a first molar LM1 of the lower teeth, and is attached to the posterior teeth section by a hook fixing member 21 of the second hook. Of course, the second hook 20b may be fixed to the second molar LM2. Accordingly, in the fourth embodiment too, the elastic member described above can be connected from the buccal side of the lower teeth to the lingual side of the upper teeth.

Next, FIGS. 8A and 8B show a fifth embodiment of the MAD and the embodiment of FIGS. 8A and 8B includes the upper frame 100 (100A), the lower frame 200 (200A), and the second hook 20 that have the same structures as those of the example shown in FIGS. 7A and 7B, but the fifth embodiment has a first hook 20a that is fixed (attached) to a specific tooth of the upper teeth.

In more detail, in the fifth embodiment, the first hook 20a is fixed to the buccal side of the front teeth section, for example, both canines of the upper teeth and is attached to the front teeth section, that is, the canines of the upper teeth by a hook fixing member 11 of the first hook. Of course, the teeth to which the first hook 20a is attached are not limited to the canines. Accordingly, in the fifth embodiment, the elastic member described above can be connected from the buccal side of the lower teeth to the labial side of the upper teeth.

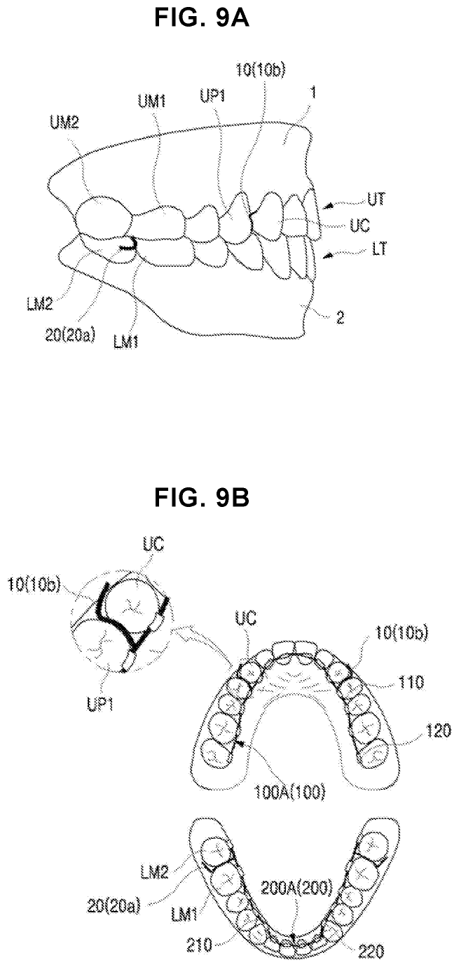

FIGS. 9A and 9B show a sixth embodiment of the MAD and the embodiment of FIGS. 9A and 9B includes the upper frame 100 (100A), the lower frame 200 (200A), and the second hook 20a that have the same structures as those of the example shown in FIGS. 5A and 5B, but, in the sixth embodiment a first hook 10b that is connected to the upper frame, that is, the upper archwire 100A is positioned at the buccal side.

In detail, the first hook 10b may be formed on a buccal side in an area from a premolar UP1 and a canine UC across the embrasure between the canine UC and the first premolar UP1 of the upper archwire 100A. Accordingly, in the sixth embodiment too, the elastic member described above is connected from the buccal side of the lower teeth to the labial side of the upper teeth.

The MAD using an upper archwire and a lower archwire is not limited to the examples described above and mandibular advancement devices with various structures not shown in FIGS. 5A to 9B may be implemented by alternately applying any one of the first hooks 10 with various structures and any one of the second hooks 20 with various structures shown in FIGS. 5A to 9B. For example, it is possible to implement an MAD including the first hook 10b of FIGS. 9A and 9B and the second hook 20 of FIGS. 5A and 5B or the first hook 10b of FIGS. 9A and 9B and the second hook 20b of FIGS. 7A and 7B.

An MAD according to the present disclosure a mandibular advancement device, that, is a hybrid-type MAD including a removable frame that is mounted on any one of upper teeth and lower teeth, for example, a splint type frame and a fixable frame that is mounted on the other one, for example, a teeth-attachable frame such as the archwire time frame exemplified in FIGS. 5A to 9B.

In more detail, another embodiment (seventh embodiment) of the MAD may include a removable frame, that is, an upper splint 100B that is mounted on upper teeth, a first hook 10 supporting an elastic member, a fixable frame, that is, a lower archwire 200A that is mounted on lower teeth, and a second hook 20 connecting the elastic member.

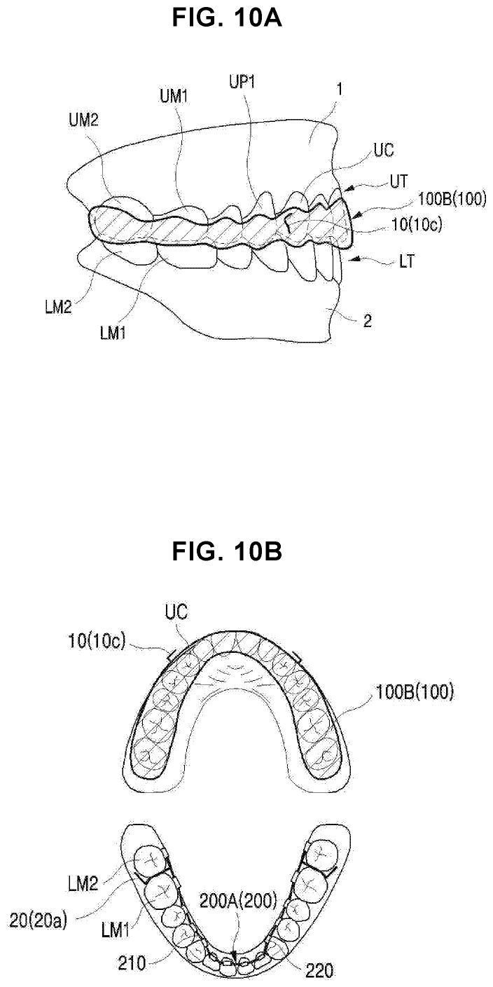

For example, as shown in FIGS. 10A and 10B, the seventh embodiment of the MAD may include an upper splint 100B that can be removably mounted on upper teeth UT, and a lower frame 200 (200A) and a second hook 20a that are the same as those in the example shown in FIGS. 6A and 6B, and a first hook 10c supporting an elastic member may be disposed at the upper splint 100B. For example, one end portion of the first hook 10c may be embedded in a side of the upper splint.

In this embodiment, the first hook 10c may be disposed in both front teeth areas (both side areas of a front teeth seat section) of the upper splint 100B, in more detail, the labial side of the upper splint 100B, but is not limited thereto and may be disposed at other positions. The structure, for example the length, the direction, or the like of the first hook 10c may be changed in various ways. The seventh embodiment of the MAD may include the second hook 20, 20b of FIGS. 5A, 5B, 7A or 7B rather than the second hook 20a of FIGS. 6A and 6B.

As another example (fourth embodiment), the MAD may include a fixable frame, that is, an upper archwire 100A that is mounted on upper teeth UT, a first hook 10 supporting an elastic member, a movable frame, that is, a lower archwire 200B that is mounted on lower teeth LT, and a second hook 20 connecting the elastic member.

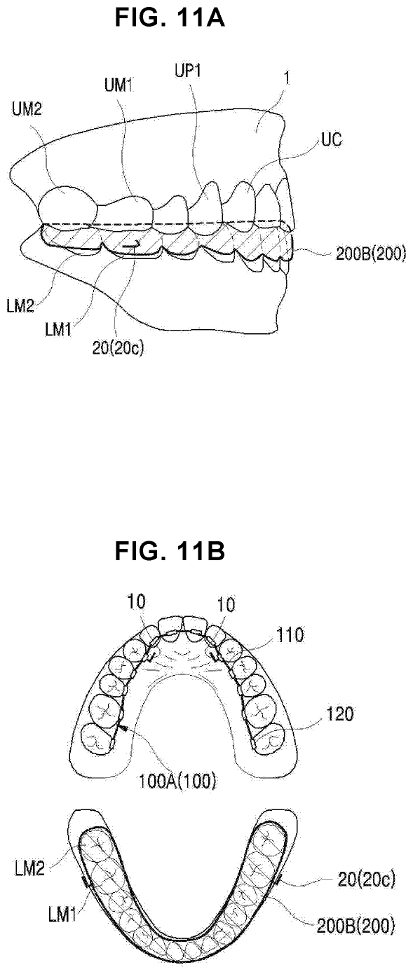

For example, as shown in FIGS. 11A and 11 B, the eighth embodiment of the MAD may include an upper archwire 100A and a first hook 10 that are fixed to upper teeth that are the same as those in the example shown in FIGS. 5A and 5B, and a lower splint 200b that are removably mounted on lower teeth LT, and a second hook 20c connecting an elastic member may be disposed at the lower splint 200B. For example, one end portion of the second hook 20c may be embedded in a side of the lower splint 200B.

In this embodiment, the second hook 20c may be disposed in both posterior teeth areas (both side areas of a posterior teeth seat section) of the lower splint 200B, in more detail, on the labial side of the lower splint 200B in the molar area, but is not limited thereto and may be disposed at other positions. The structure, for example the length, the direction, or the like of the second hook 20c may be changed in various ways. The eighth embodiment of the MAD may include the first hook 10a, 10b of FIG. 8A, 8B, 9A or 9B rather than the first hook 10 of FIGS. 5A and 5B.

As another example (ninth embodiment), the MAD may include an upper frame 100 including an upper archwire 100A that is mounted on upper teeth UT in a fixed type and an upper splint 100C in which the maxillary arch is fitted, a first hook 10 supporting an elastic member, a second archwire 200A that is fixed to lower teeth LT, and a second hook 20 connecting the elastic member.

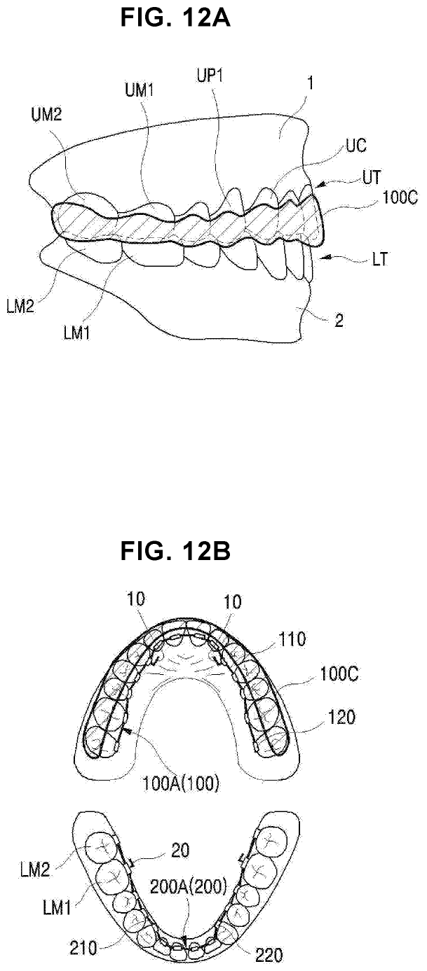

For example, as shown in FIGS. 12A and 12B, the upper archwire 100A is fixed to the lingual side of the upper teeth UT and the lingual side of the upper splint 100C is open to prevent interference between the upper archwire 100A and the upper splint 100C. The first hook 10 may be disposed in both front teeth areas of at least one of the upper archwire 100A and the upper splint 100C.

The first hook 10, as in the structure shown in FIGS. 5A and 5B, may be formed on the lingual side of the upper archwire 100A, but is not limited thereto, and for example, may be formed at a proximal area in comparison to the second hook 20 such as the labial side of the upper splint 100C. The upper splint 100C may have a structure that covers the labial side, the buccal side, the occlusion surface, and the incisal margin of the upper teeth and is open on the lingual side. The second hook 20 has the same structure as that exemplified in FIGS. 5A and 5B, but is not limited thereto, and may be changed in other structures such as the structure 20a, 20b exemplified in FIG. 6A, 6B, 7A or 7B.

As another example (tenth embodiment), the MAD may include an upper archwire 100A that is mounted on upper teeth in a fixed type, a first hook 10 supporting an elastic member, a lower frame 200 including a lower archwire 200A that is mounted on lower teeth in a fixed type and a lower splint 200C in which the mandibular arch is fitted, and a second hook 20 connecting the elastic member.

For example, as shown in FIGS. 12A and 12B, the lower archwire 200A is fixed to the lingual side of the lower teeth and the lingual side of the lower splint 100C is open to prevent interference between the lower archwire 200A and the lower splint 200C.

The first hook 10, as in the structure shown in FIGS. 4A and 4B, may be formed on the lingual side of the upper archwire 100A, but is not limited thereto, and for example, other structures such as the structure 10a, 10b shown in FIGS. 7A, 7B, 8A or 8B are possible. The lower splint 100C may have a structure that covers the labial side, the buccal side, the occlusion surface, and the incisal margin of the lower teeth and is open on the lingual side. The second hook 20 may be disposed in both posterior teeth areas of at least one of the lower splint 200C and the lower archwire 200A.

For example, the second hook 20, as in the structure shown in FIGS. 5A and 5B, may be formed on the lingual side of the lower archwire 200A, but is not limited thereto, and for example, may be formed at a distal area in comparison to the first hook 10 such as the buccal side of the lower splint 200C.

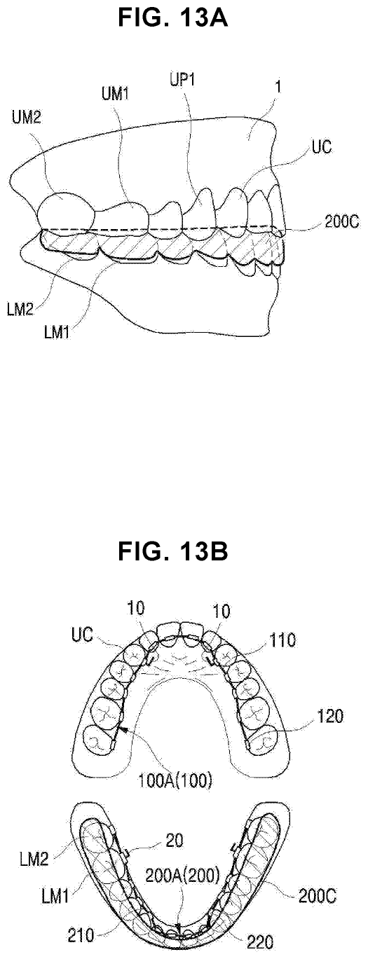

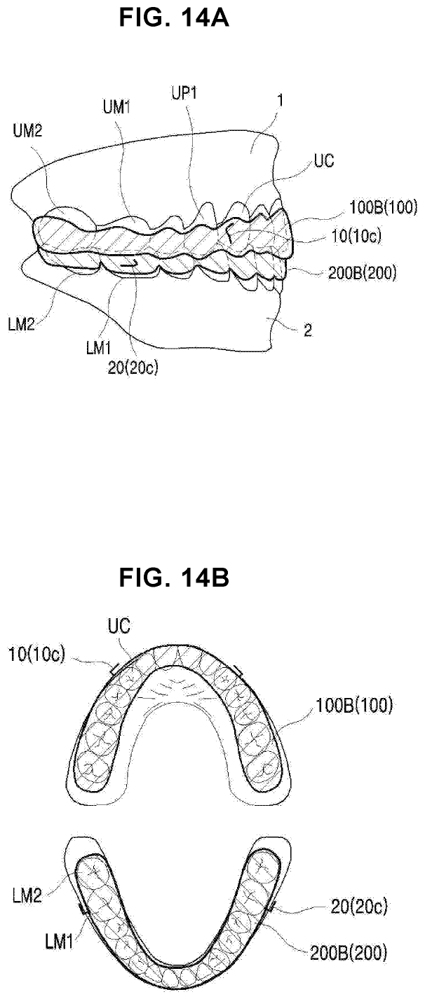

As in the examples shown in FIGS. 10A to 13B, when a splint type frame is applied to only any one of upper teeth and lower teeth, the mandible can smoothly slide forward, and an occlusal clearance can be reduced and it is possible to more comfortably use the device as compared with when a splint type frame is applied to both of upper teeth and lower teeth as in the examples of FIGS. 14A, 14B and 15.

Next, the MAD shown in FIGS. 14A and 14B include the removable upper frame (upper splint 100B) and lower frame (lower splint 200B) shown in FIGS. 3A and 3B, a first hook 10c is disposed at the upper splint 100B, and a second hook 20c is disposed at the lower splint 200B. Accordingly, an elastic member is easily attached/detached and a teeth-attachable MAD can be used.

Referring to FIG. 15, the upper frame may include an upper splint 100D having a shape in which the occlusion sides, that is, the bottom sides of the molar seat sections in which posterior teeth, that is, molars UM1 and UM2 of upper teeth are fitted are bored.

Although only the bottom sides of the molar seat sections are open in FIG. 15, the present disclosure is not limited thereto, and for example, the most distal tooth area of upper teeth, in detail, at least a portion from the second molar area and a premolar area may be open.

Referring to FIG. 15, the lower frame may include a lower splint 200D having a shape in which the occlusion sides, that is, the top sides of the molar seat sections in which posterior teeth, that is, molars LM1 and LM2 of lower teeth are fitted are bored.

Although only the top sides of the molar seat sections are open in FIG. 15, the present disclosure is not limited thereto, and for example, the most distal tooth area of lower teeth, in detail, at least a portion from the second molar area and a premolar area may be open.

When any one of the upper splint 100D and the lower splint 200D is bored at the posterior occlusion sides, as in the example of FIG. 15, the occlusal clearance can be reduced and the device can be more comfortably used, considering movement of the temporomandibular joint.

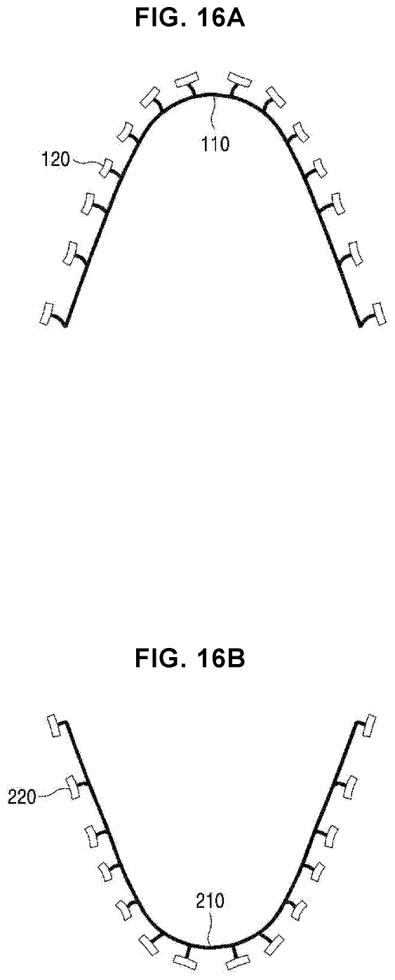

The upper archwire that is fixed to the maxillary arch may be configured to pass the necks of upper teeth or the height of the gums. In more detail, the lingual archwire 110 of the upper archwire, as in the example shown in FIGS. 2A and 2B, may be directly fixed to the wire fixing members 120 of upper teeth across the lingual side of the upper teeth, but is not limited thereto, and may be indirectly fixed to the wire fixing members 120 of upper teeth, as in the example shown in FIG. 16A. For example, the lingual archwire 110 of the upper archwire may be configured to pass the necks of upper teeth or the lingual side of the gums with a distance (height difference) from a virtual line connecting the wire fixing members 120 so that interdental cleaning is easy.

The lower archwire that is fixed to mandibular arch may also be configured to pass the necks of lower teeth or the height of the gums. In more detail, the lingual archwire 210 of the lower archwire, as in the example shown in FIGS. 2A and 2B, may be directly fixed to the wire fixing members 120 of lower teeth across the lingual side of the upper teeth, but is not limited thereto, and may be indirectly fixed to the wire fixing members 220 of lower teeth, as in the example shown in FIG. 16B. For example, the lingual archwire 210 of the lower archwire may be configured to pass the necks of lower teeth or the lingual side of the gums with a distance (height difference) from a virtual line connecting the wire fixing members 120 of lower teeth so that interdental cleaning is easy.

Although preferred embodiments of the present disclose were described above, it would be apparent to those skilled in the art that the present disclosure may be achieved in other specific types without departing from the scope or spirit other than the embodiments described above.

Accordingly, the embodiments described above should be considered as being exemplifying rather than limiting, so the present disclosure may be changed within the range of the claims and the equivalent range without being limited to the above description.

What is claimed is:

1. A mandibular advancement device comprising:
a first hook to which a first end of an elastic member providing a mandible traction force for advancing the mandible can be connected and that supports the elastic member;
an upper frame that can be mounted on upper teeth to distribute a traction support force, which is applied to the first hook by the elastic member, to a maxilla;
a second hook to which a second end of the elastic member can be connected and that transmits the mandible traction force applied by the elastic member to the mandible at a distal location in comparison to the first hook; and
a lower frame that can be mounted on lower teeth under the upper frame to distribute the mandible traction force, which is applied to the second hook, to the mandible.

2. The mandibular advancement device of claim 1, wherein the upper frame can be mounted on the upper teeth in a fixed type or a removable type to fasten the upper teeth; and the lower frame can be mounted on the lower teeth in a fixed type or a removable type to fasten the lower teeth.

3. The mandibular advancement device of claim 1, wherein the first hook is disposed at the upper frame; and the second hook is disposed at the lower frame.

4. The mandibular advancement device of claim 1, wherein the upper frame includes an upper archwire that is mounted in a fixed type on a maxillary arch (superior dental arch).

5. The mandibular advancement device of claim 4, wherein the first hook is disposed at both front teeth areas of the upper archwire.

6. The mandibular advancement device of claim 5, wherein the upper archwire is a lingual archwire that can be disposed on a lingual side of the maxillary arch; and the first hook is formed on a lingual side or a palatal side of the upper archwire or is formed on a buccal side across an embrasure between a canine and a first premolar of the upper archwire.

7. The mandibular advancement device of claim 1, wherein the upper frame includes an upper splint that is fitted on a maxillary arch (superior dental arch).

8. The mandibular advancement device of claim 7, wherein the first hook is disposed at both front teeth areas of the upper splint.

9. The mandibular advancement device of claim 7, wherein the upper splint has a shape in which occlusion sides of molar seat sections in which the upper molars are fitted are bored such that occlusal surfaces of the upper molars are exposed.

10. The mandibular advancement device of claim 4, wherein the lower frame includes at least one of a lower archwire that is mounted in a fixed type to a mandibular arch and a lower splint in which the mandibular arch is fitted.

11. The mandibular advancement device of claim 10, wherein the second hook is disposed at both posterior teeth seat sections of the lower splint.

12. The mandibular advancement device of claim 10, wherein the second hook is disposed at both posterior teeth areas of the lower archwire.

13. The mandibular advancement device of claim 12, wherein the lower archwire is a lingual archwire that can be disposed at a lingual side of the mandibular arch; and the second hook includes a lingual hook formed on a lingual side of the lower archwire or a buccal hook formed on a buccal side across an embrasure of posterior teeth of the lower archwire.

14. The mandibular advancement device of claim 10, wherein the lower splint has a shape in which occlusion sides of molar seat sections in which the lower molars are fitted are bored such that occlusal surfaces of the lower molars are exposed.

15. The mandibular advancement device of claim 1, wherein the lower frame includes a lower archwire that is mounted in a fixed type at a lingual side of lower teeth and a lower splint in which a mandibular arch is fitted;
a lingual side of the lower splint is open to prevent interference between the lower splint and the lower archwire; and
the second hook is disposed at both posterior teeth areas of at last one of the lower splint and the lower archwire.

16. The mandibular advancement device of claim 1, wherein the upper frame includes an upper archwire that is mounted in a fixed type at a lingual side of a maxillary arch and an upper splint in which the maxillary arch is fitted
a lingual side of the upper splint is open to prevent interference between the upper splint and the upper archwire; and
the first hook is disposed at both front teeth areas of at last one of the upper splint and the upper archwire.

17. The mandibular advancement device of claim 1, wherein the elastic member can be attached and detached to and from the first hook and the second hook.
